(19) United States
(12) Patent Application Publication
(10) Pub. No.: US 2009/0036731 A1
(43) Pub. Date: Feb. 5, 2009

(54) Apparatus and method of enhancing sexual satisfaction via an implantable vibrating device

(76) Inventor: Michael Salzhauer, Bal Harbor, FL (US)

Correspondence Address: ROBERT M. SCHWARTZ, P.A., P.O. BOX 221470, HOLLYWOOD, FL, 33022, US

(21) Appl. No.: 12/184,361

(22) Filed: Aug. 1, 2008

Related U.S. Application Data
(63) Continuation of application No. 60/953,383, filed on Aug. 1, 2007.

(51) Int. Cl. A61F 5/00; A61H 1/00

(52) U.S. Cl. 600/38; 601/46

(57) ABSTRACT

A device and method constructed and arranged to be implanted under the skin of a user that provides vibrational energy to provide sexual enhancement or treat sexual dysfunction.

### INDEX TO RELATED APPLICATION

This application claims the benefit of U.S. Provisional Patent Application 60/953,383, filed Aug. 1, 2007, the disclosure of which is incorporated herein by reference in its entirety.

### BACKGROUND OF THE INVENTION

There are a number of diseases/conditions which directly affect the sexual lives of humans and animals. These include orgasmic disorders, lack of interest in sex, erectile dysfunction in males, hormonal and physiological imbalances in females, among others. In addition there are a number of diseases/conditions which indirectly affect the sexual lives of humans as well as certain therapeutic treatments that effect sexual abilities and functions. There some few pharmaceutical agents available to treat sexual disorders and others in clinical development.

However, pharmaceutical agents carry the risk of undesired side effects. There is a need to provide a physical device that may treat and/or ameliorate sexual disorders.

### BRIEF SUMMARY OF THE INVENTION

The present invention is a device for providing sexual enhancement and for treating, preventing, and ameliorating sexual dysfunction.

In one embodiment, the present invention is an implantable device for sexual enhancement or treating sexual dysfunction comprising:
a. an implantable shell;
b. an electrical source;
c. a vibrational motor powered by said electrical source;
whereby said devices constructed and arranged for being implanted internally below the surface of the skin.

Preferably, the implantable shell is formed of silicone or any other acceptable implantable material. Implantable material relates to materials that may be surgically or otherwise inserted below the skin of a patient.

The device is constructed and arranged to be implanted below the surface of the skin. Therefore, must be sufficiently small to accommodate implantation.

The may have a rechargeable battery and provides for an external access of recharging means for said electrical source.

The electrical source may be direct connection recharge by way of access to wiring outside the body, or may be rechargeable by induction charging.

The device of is started when the device electrical source is actuated. Actuated may be by any means and may include but would not be limited to physical on/off switch activation or by wireless transmission.

Additionally contemplated is a method for providing sexual enhancement or treating, preventing, or ameliorating sexual dysfunction having the steps of:
a. providing a device according to claim 1;
b. performing a surgical incision suitable for implantation of said device;
c. surgically implanting said device in a region suitable for treating sexual dysfunction;
d. closing said surgical incision and allowing for any desired external access in said closing;
e. actuating electrical power to said device, whereby said actuation initiates the motion of a vibrational motor.

### BRIEF DESCRIPTION OF THE DRAWINGS

FIG. 1 is a front view showing possible implantation sites for the present invention in a male.

FIG. 2 is a front view showing possible implantation sites for the present invention in a female.

FIG. 3 is an enlarged front view showing possible implantation sites for the present invention in a male.

FIG. 4 is an enlarged front view showing possible implantation sites for the present invention in a female.

FIG. 5 is a schematic showing the components of the invention.

FIG. 6 is a schematic showing the components of the invention.

FIG. 7A is a schematic showing an implanted device with exterior on/off switch.

FIG. 7B is a schematic showing an implanted device with rechargeable exterior battery recharge connections.

FIG. 7C is a schematic showing an implanted device with exterior wiring for battery recharge connections.

FIG. 7D is a schematic showing an implanted device with exterior connections for battery recharge connections.

FIG. 8D is a schematic showing an implanted device with wireless battery recharge connections.

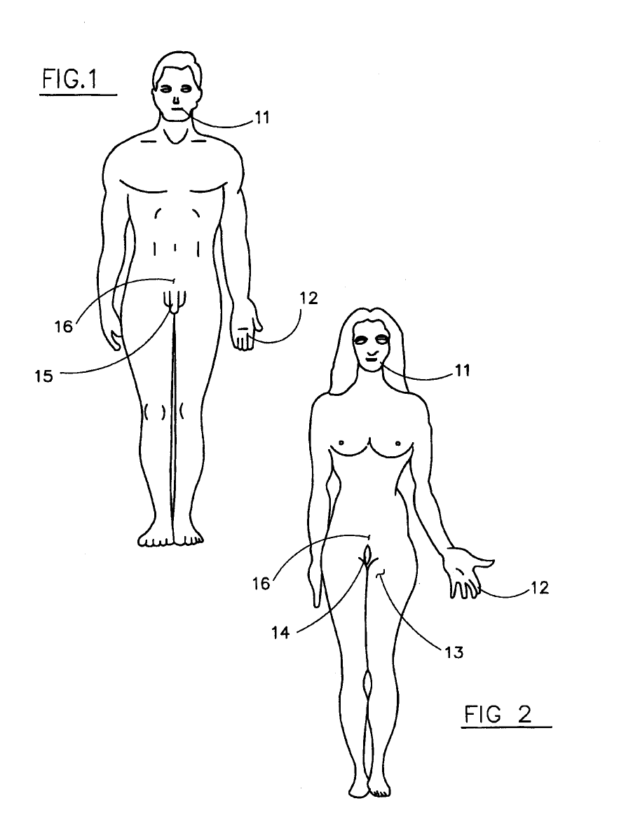

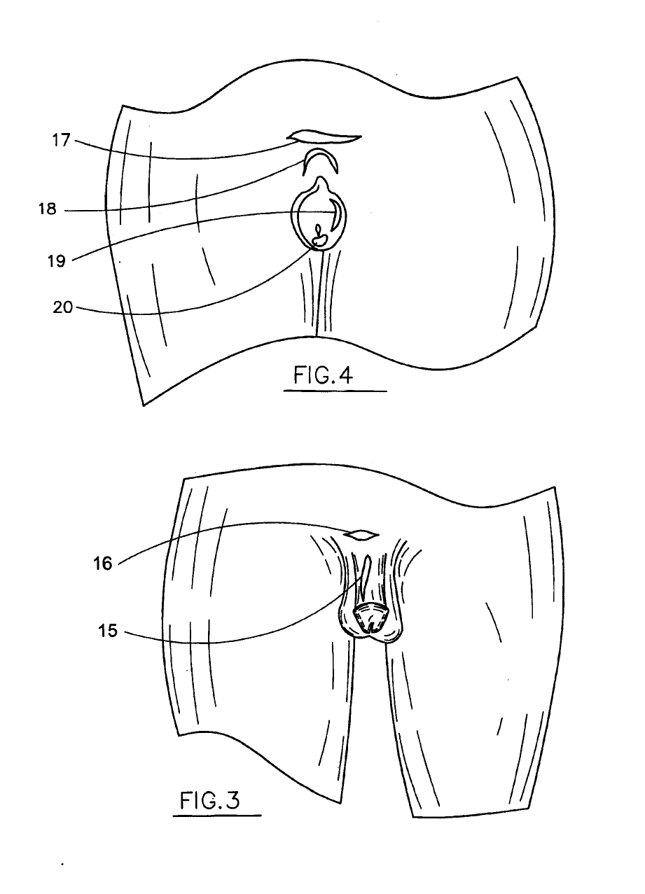

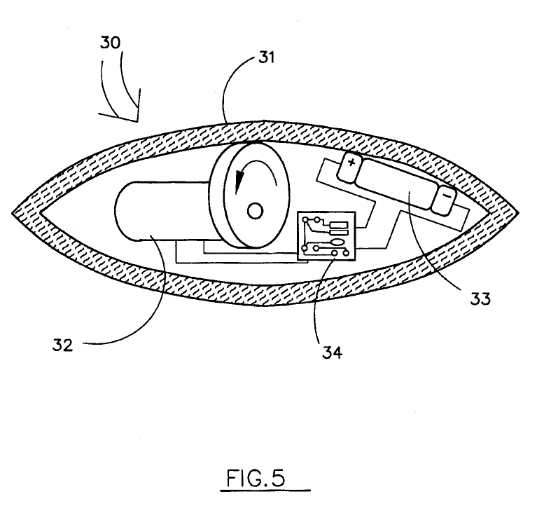

### DETAILED DESCRIPTION OF THE PREFERRED EMBODIMENTS

Device 30 has a shell 31. Shell 31 surrounds a hollow cavity within device 30 in which components are housed. Vibrational motor 32 receives power from battery 33 and is regulated by control circuit board 34. In one embodiment, device 40

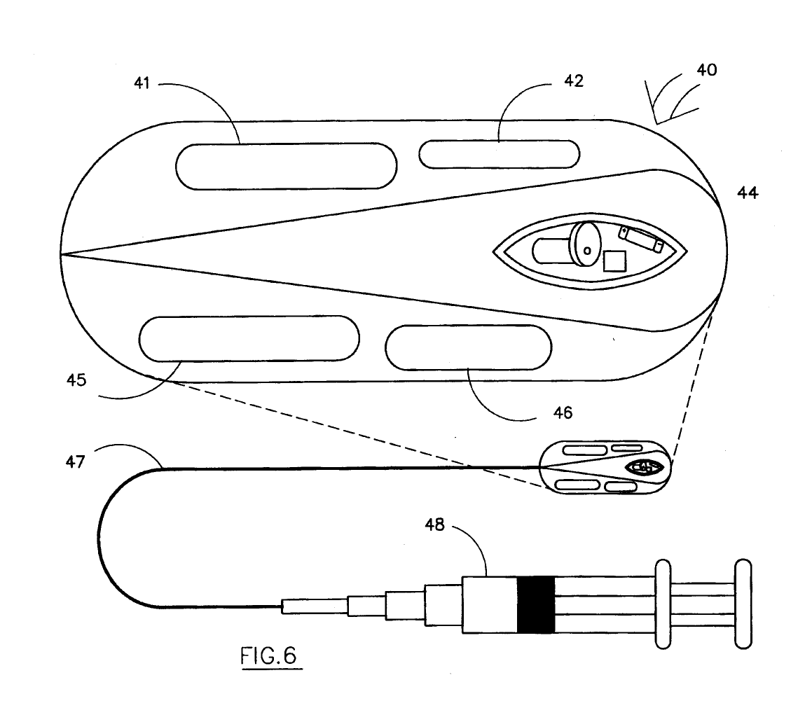

In embodiment as shown in FIG. 6 device 40 of the present invention may be configured to fit within existing implantable medical devices. Existing implant 44 has a surface of silicon 41 and they slip plane at 42 that maintains the position of the implant under the skin surface. A self sealing valve 45 of existing devices allows for devices to be filled with various liquids through fill tube 47 by means of plunger 48.

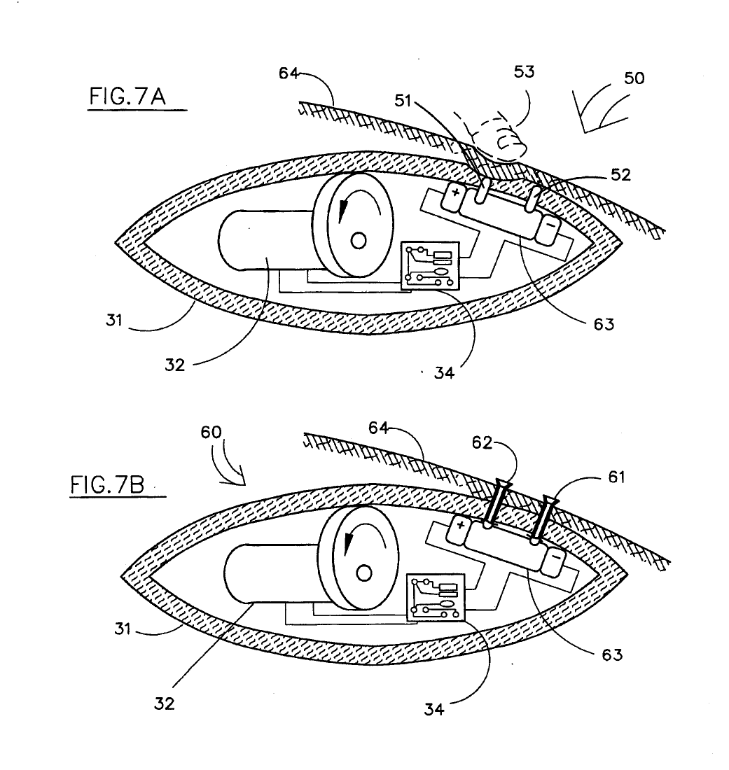

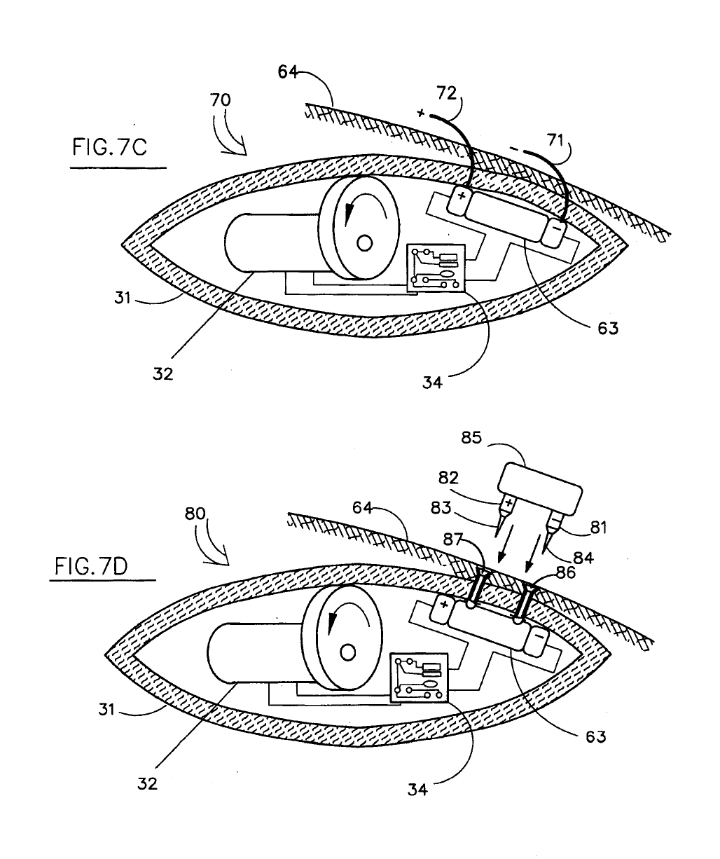

In an embodiment shown in FIG. 7A, configuration 50 has device 30 is implanted below skin surface 64. A finger 53 presses on skin surface 64 and manually activates a switch actuator 51 to start the vibrational motion of motor 32. Switch 52 is a switch that turns off the power to motor 52.

Configuration 60, shown in FIG. 7B, provides for external ports 61 and 62 visible above the surface of skin 64. Ports 64 may be any single or combination of on/off switches, battery recharge access ports.

Configuration 70, shown in FIG. 7C has a positive electrical lead 72 and a negative electrical lead 71 extending though skin surface 64 such that they are accessible outside the body.

Configuration 80, shown in FIG. 7D, provides ports 86 and 87 extend from device 30 outward through skin surface 64 and are accessible from outside the body. Ports 86 and 87 are, in this embodiment, female sockets constructed and arranged corresponding male sockets 83 and 84 that are positive lead 82 and negative lead 81 of a power recharge device 85.

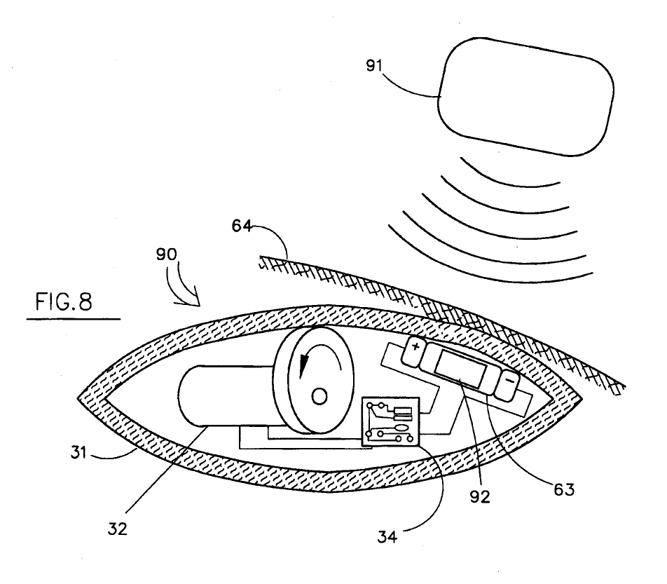

Configuration 90, as shown in FIG. 8, provides for induction charging of batteries 63. An electrical induction charging transmitter 91 is outside the body and above skin surface 64. An induction charging receiver 92 is placed within the cavity of device 30 and is constructed to receive a wireless transmission from charging transmitter 91.

As discussed herein, device refers to any of the configured devices disclosed in described and is not limited to any single embodiment.

The device of the present invention is constructed and arranged to be implanted underneath the skin of a user. Implantation sites may include but would not be limited to lips 11, fingers 12, groin area 13, labia 14, penile shaft 15, and suprapubic region 16.

When implanted in the female pubic region the device may be implanted in any one of the low suprapubic region 17, periclitoralhood region 18, labia menora 19, and/or the intravaginal region 20.

Additionally contemplated, would be the implantation of more than one device of the present invention.

The device of the present invention commences its vibrational motion by means of actuation of an electrical switch. The actuation may occur by any desired means in an electrical actuation may be obtained. In one embodiment a manual switch 51 is accessible from the outer portion of skin surface 64. Although the embodiment depicted in FIG. 7A provides for a switch on 51 and a switch off 52, it is contemplated that the on and off may be accomplished through actuation of a single switch. Additionally contemplated in the present invention would be actuation of the motor through interconnection of either electrical leads 71 and 72 or through ports 86 and 87. An external on off mechanism may be utilized in conjunction with any of the battery or electrical recharge mechanisms. Also contemplated in the present invention would be a voice actuated on off switch whereby by the voice actuation unit is located outside surface 64 and signal is transmitted through any of the aforementioned electrical leads 71 and 72 or ports 86 and 87. Alternatively, a voice actuator may be located within shell 31.

Electrical leads 71 and 72 or ports 86 and 87, may additionally provide access to the unit whereby the electrical energy may be regulated and thus the vibrational motion of motor 32 may be regulated as desired.

The device of the present invention is surgically implanted beneath skin surface 64 by means commonly known. If desired, a medical professional performing the surgery will provide for external leads 71 and 72 or any other necessary external devices to be accessible through skin surface 64 after the device has been implanted.

The device has a vibrational motor 32 that provides vibrational motion to the unit when the unit is actuated. The device of the present invention provides a physical stimulation by which sexual stimulation and enhancement and/or sexual disorders in males and females may be treated. The device is advantageous in that it does not require chemical or pharmaceutical treatment for sexual dysfunction. Many individuals with sexual dysfunction disorders may be able to achieve satisfactory treatment by utilization of the device the present invention without the need of additional chemical or pharmaceutical treatment.

Some persons may have disorders of severity such that the physical stimulation provided in the device of the present invention may not provide an adequate level of treatment. In such individuals, it may be desirable to employ the device of the present invention in conjunction with conventional chemical and or pharmaceutical treatment. It is further contemplated that if such chemical or pharmaceutical treatment were to be used in conjunction with the device of the present invention, such treatment may be able to proceed and be effective at reduced levels that are commonly utilized.

In one embodiment, the device may be actuated by wireless remote control. The wireless actuation may include but would not be limited to Bluetooth or Rf transmission of an actuating signal to the device.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An implantable device for treating sexual dysfunction comprising:
a. an implantable shell;
b. an electrical source;
c. a vibrational motor powered by said electrical source;
whereby said devices constructed and arranged for being implanted internally below the surface of the skin.

2. The device of claim 1 wherein the implantable shell is formed of silicone.

3. The device of claim 1 wherein the devices constructed and arranged to be implanted below the surface of the skin.

4. The device of claim 1 wherein the electrical source is a rechargeable battery,

5. The device of claim 1 the device provides for an external access of recharging means for said electrical source.

6. The device of claim 1 wherein said electrical source is rechargeable by induction charging.

7. The device of claim 1 wherein said electrical source is actuated by wireless transmission.

8. A method for providing sexual enhancement or treating, preventing, or ameliorating sexual dysfunction having the steps of:
a. providing a device according to claim 1;
b. performing a surgical incision suitable for implantation of said device;
c. surgically implanting said device in a region suitable for treating sexual dysfunction;
d. closing said surgical incision and allowing for any desired external access in said closing;
e. actuating electrical power to said device, whereby said actuation initiates the motion of a vibrational motor.
